(19) United States
(12) Patent Application Publication
(10) Pub. No.: US 2026/0306207 A1
(43) Pub. Date: Oct. 8, 2026

(54) CLAMPING DEVICE AND TOOL HOLDER

(71) Applicant: Aesculap AG, Tuttlingen (DE)

(72) Inventor: Eda Ay-Becker, Aach (DE)

(21) Appl. No.: 19/641,926

(22) Filed: Apr. 8, 2026

Related U.S. Application Data
(63) Continuation of application No. PCT/EP2024/077756, filed on Oct. 2, 2024.

(30) Foreign Application Priority Data
Oct. 9, 2023 (DE) 10 2023 127 412.0

(51) Int. Cl. A61B 50/20

(52) U.S. Cl. CPC A61B 50/20

(57) ABSTRACT

A clamping device supports a surgical tool in a clamped state on a perforated plate. The clamping device has a clamping apparatus and a fastening apparatus. The clamping apparatus has a number of clamping springs, each having a clamping limb for clamping the surgical tool. The clamping springs extend from a base of the clamping apparatus in a longitudinal direction. The clamping limbs define an insertion opening for inserting the surgical tool into the clamping apparatus. The fastening apparatus has a number of fastening springs, each having a fastening limb for fastening the clamping device to the perforated plate. The fastening springs extend from a base of the fastening apparatus in the longitudinal direction. Each fastening limb has a fastening unit for fastening the clamping device to the perforated plate. The clamping apparatus and fastening apparatus are connected to one another by a frictional, integral and/or interlocking connection.

### CROSS-REFERENCE TO RELATED APPLICATIONS

This application is a continuation under 35 U.S.C. § 365 of International Application No. PCT/EP2024/077756, filed on Oct. 2, 2024, and claims priority to German Application No. 10 2023 127 412.0, filed on Oct. 9, 2023. The contents of International Application No. PCT/EP2024/077756 and German Application No. 10 2023 127 412.0 are incorporated by reference herein in their entireties.

### FIELD

The present disclosure relates to a clamping device and a tool holder with the clamping device.

### BACKGROUND

Clamping devices are frequently used to support surgical tools in a clamped position on a perforated plate.

DE 10 2021 106 109 A1, DE 10 2021 106 110 A1 and DE 10 2021 106 111 A1 each show clamping devices that are designed in one piece.

### SUMMARY

It is the object of the present disclosure to provide a clamping device which has improved properties, in particular is economical to produce. Furthermore, the object of the present disclosure is to provide a tool holder that has such a clamping device.

A clamping device according to the present disclosure is designed for supporting a surgical tool in a clamped state on a perforated plate. The clamping device has a clamping apparatus and a fastening apparatus. The clamping apparatus has a number, in particular 1 to 10, preferably 1 to 4, of clamping springs, each with a clamping limb for clamping the surgical tool. The number of clamping springs extends from a base of the clamping apparatus in a longitudinal direction of the clamping device. The clamping limbs define an insertion opening for inserting the surgical tool into the clamping apparatus. The fastening apparatus has a number, in particular 1 to 10, preferably 1 to 4, of fastening springs, each with a fastening limb for fastening the clamping device to the perforated plate. The number of fastening springs extends from one base of the fastening apparatus in the longitudinal direction of the clamping device. Each fastening limb has a fastening unit for fastening the clamping device to the perforated plate, in particular in a detachable manner. The clamping apparatus and the fastening apparatus are connected to one another by means of a frictional, integral and/or interlocking connection. The clamping apparatus and the fastening apparatus can each be formed from a bent sheet of metal.

The frictional, integral and/or interlocking connection advantageously allows for a modular design of the clamping device. For example, the modular design allows the fastening apparatus and/or the clamping device to be used as standardized individual components in other devices. This allows the fastening apparatus and/or the clamping apparatus to be manufactured in a larger quantity than is required for the production of clamping devices, thereby reducing the cost per clamping device, and the clamping device is therefore economical to manufacture.

Another aspect of the clamping device can be that by forming the clamping apparatus and the fastening apparatus each from a bent sheet of metal, the manufacture of the clamping device is simplified and economical. In particular, identical or similar bending tools can be used for manufacturing the clamping apparatus and the fastening apparatus, which can save further costs.

The surgical tool can be an instrument head, in particular a drilling or milling tool with or without a tool shank. The surgical tool can be designed to be inserted into a hand-held surgical instrument.

The surgical tool can be inserted into the clamping device by inserting the surgical tool into the insertion opening. The surgical tool can be brought into the clamped state by inserting it into the clamping device. In the clamped state, each clamping spring can exert a spring force on the surgical tool, which presses the surgical tool against the other clamping springs. The spring force can be generated, for example, by pre-tensioning the number of clamping springs when the surgical tool is inserted into the clamping device.

Each clamping spring can have an insertion ramp for inserting the surgical tool into the clamping device. The insertion ramps can limit the insertion opening. Insertion can be achieved by pressing the surgical tool against the insertion ramps, which causes the surgical tool to slide along the insertion ramps and pre-tension the number of clamping springs until the clamped state of the surgical tool is reached.

The surgical tool can be inserted into the clamping device by a directed movement of the surgical tool parallel to, or in particular opposite to, the longitudinal direction. The longitudinal direction can run parallel to a longitudinal axis of the clamping device.

Each clamping limb can have a contact portion for frictional and/or interlocking contact of the surgical tool in the clamped state. Each contact section can protrude radially inwards.

The number of clamping springs can be arranged, in particular evenly and/or circumferentially, distributed around the longitudinal axis.

The number of fastening springs can be arranged, in particular evenly and/or circumferentially, distributed around the longitudinal axis.

The base of the clamping apparatus and/or the base of the fastening apparatus can have a portion that is designed as a circular disk. The base of the clamping apparatus and/or the base of the fastening apparatus can be arranged perpendicular to the longitudinal direction.

The base of the clamping apparatus and/or the base of the fastening apparatus can be arranged at a first end of the clamping device. The base of the clamping apparatus and/or the base of the fastening apparatus can form the first end of the clamping device. The base of the clamping apparatus and/or the base of the fastening apparatus can serve as a limit to the insertion depth of the surgical tool into the clamping device.

The insertion opening can be arranged at a second end of the clamping device opposite the first end.

The fastening unit can be designed to fasten the clamping device to the perforated plate by means of a frictional, integral and/or interlocking connection. The fastening unit can have a locking element. The locking element can be designed to lock with the perforated plate. The locking element can be designed to fix the clamping device to the perforated plate by means of a locking connection formed between the locking element and the perforated plate.

To attach the clamping device to the perforated plate, the fastening apparatus can be displaced by pre-tensioning the number of fastening springs in such a way that the fastening unit can be inserted at least partially into a hole of the perforated plate, wherein by releasing the pre-tensioned number of fastening springs, the fastening apparatus is repositioned in such a way that the clamping device is fastened to the perforated plate by means of the fastening apparatus.

In particular, the clamping apparatus and the fastening apparatus can only be connected to one another by means of a frictional, integral and/or interlocking connection.

The clamping apparatus and/or the fastening apparatus can be formed from a material that contains iron. The clamping apparatus and/or the fastening apparatus can be made of a flexible metal, preferably a spring steel, for example formed from the material 1.4310.

The sheet metal from which the clamping apparatus is formed can have a width and/or a length that is more than five times, in particular ten times, the thickness of the sheet metal from which the clamping apparatus is formed.

The sheet metal from which the fastening apparatus is formed can have a width and/or a length that is more than five times, in particular ten times, the thickness of the sheet metal from which the clamping apparatus is formed.

In a further development of the clamping device, the sheet metal from which the clamping apparatus is formed and the sheet metal from which the fastening apparatus is formed are made of the same material. Additionally or alternatively, the sheet metal from which the clamping apparatus is formed and the sheet metal from which the fastening apparatus is formed have the same thickness.

Advantageously, forming the sheet metal of the clamping apparatus and forming the sheet metal of the fastening apparatus from the same material can facilitate the creation of the connection between the clamping apparatus and the fastening apparatus. Preferably, the clamping apparatus and the fastening apparatus can be very easily glued, clamped or welded together. In particular, welding the clamping apparatus to the fastening apparatus can be simplified due to the same melting temperature and the same physical and chemical properties of the material.

Further cost reductions can be achieved in that the sheet metal of the clamping apparatus and the sheet metal of the fastening apparatus are formed from the same material and/or have the same thickness. In particular, the clamping apparatus and the fastening apparatus can be made from the same sheet metal component, especially the same sheet metal component. This allows the sheet metal required for the manufacture of the clamping device to be standardized, thereby achieving further cost reductions.

In a further development of the clamping device, the clamping apparatus and the fastening apparatus are separately designed components that are connected to one another by means of a frictional, integral and/or interlocking connection. The fastening apparatus can be designed in one piece. The clamping apparatus can be designed in one piece.

In a further development of the clamping device, the base of the clamping apparatus and the base of the fastening apparatus are connected to one another by means of a frictional, integral and/or interlocking connection. Advantageously, this allows additional portions for creating the frictional, integral and/or interlocking connection to be omitted, which yields a particularly compact design of the clamping device.

In a further development of the clamping device, the clamping apparatus and/or the fastening apparatus are designed as a stamped and bent part. Advantageously, such a design of the clamping apparatus and/or the fastening apparatus enables economical production of the clamping device.

In a further development of the clamping device, the clamping apparatus has a first locking element. The fastening apparatus can have a second locking element corresponding to, preferably complementary to, the first locking element. The interlocking connection is a locking connection formed by the first locking element and the second locking element. The first locking element can have a locking opening. The second locking element may have a locking lug. The locking lug can engage in the locking opening while forming the locking connection.

In a further development of the clamping device, the clamping apparatus has a number of bent bending tabs. The interlocking connection is formed by gripping the fastening apparatus with the bent bending tabs of the clamping apparatus. Additionally or alternatively, the fastening apparatus has a number of bent bending tabs. The interlocking connection is formed by gripping the clamping apparatus with the bent bending tabs of the fastening apparatus. The interlocking connection can be an overlapping of the clamping apparatus and the fastening apparatus with each other.

In a further development of the clamping device, the interlocking connection is formed by means of a clinch connection. The clinch connection can also be called a pressure connection, press-joining connection or Toxen connection.

In a further development of the clamping device, the interlocking connection is formed by means of a deep-drawn connection. The clamping apparatus can have a through-hole. A portion of the fastening apparatus can be driven through the through-hole in the clamping apparatus by means of deep drawing. The portion of the fastening apparatus driven through the through-hole in the clamping device can be deformed in such a way that the portion of the fastening apparatus at least partially rests against the clamping apparatus while forming the interlocking connection. The base of the clamping apparatus can have a through-hole. The base of the fastening apparatus can have the portion.

Alternatively, the fastening apparatus can have a through-hole. A portion of the clamping apparatus can be driven through the through-hole in the fastening apparatus by means of deep drawing. The portion of the clamping apparatus driven through the through-hole in the fastening apparatus can be deformed in such a way that the portion of the clamping apparatus at least partially rests against the fastening apparatus while forming the interlocking connection. The base of the fastening apparatus can have a through-hole. The base of the clamping apparatus can have the portion.

In a further development of the clamping device, the interlocking connection is formed by means of an upset connection.

In a further development of the clamping device, the integral connection is formed by means of an adhesive connection, a welded connection, in particular a spot welded connection, and/or a soldered connection.

In a further development of the clamping device, the fastening unit has a first projection and a second projection. The fastening unit is designed to accommodate the perforated plate between the first projection and the second projection for fastening the clamping device to the perforated plate, in particular while forming an interlocking connection.

The first projection and the second projection can be positioned opposite each other. The first projection and the second projection can be arranged adjacent to one another, in particular in the longitudinal direction. The first projection and the second projection can extend in a direction perpendicular to the longitudinal direction. The first projection and the second projection can extend at least partially radially outwards, in particular be curved radially outwards.

The value of the distance between the first projection and the second projection can be equal to or greater than the value of the thickness of the perforated plate. The fastening unit can be designed to be passed at least partially through a hole in the perforated plate, so that in a fixed state of the clamping device on the perforated plate the perforated plate is arranged between the first projection and the second projection.

A tool holder according to the present disclosure for supporting a surgical tool in a designated position has a previously described clamping device and the perforated plate with a hole. The clamping device is fastened to the hole in the perforated plate by means of the fastening unit.

The hole can be designed to allow the surgical tool to be inserted into the clamping device. The insertion opening can be arranged inside the hole.

The fastening unit can be passed at least partially through the hole in the perforated plate so that the perforated plate is arranged between the first projection and the second projection for the purpose of fastening the clamping device to the perforated plate. The first projection can be arranged on the top side of the perforated plate, and the second projection can be arranged on the bottom side of the perforated plate.

In a further development of the tool holder, the perforated plate has a plurality of teeth that define the hole. Each fastening limb is arranged between two adjacent teeth. Advantageously, the teeth can secure the clamping device against unwanted twisting.

In a further development of the tool holder, the tool holder has an additional perforated plate to secure the clamping device against rotation. The second perforated plate is arranged parallel to and offset from the first perforated plate.

The perforated plate and the next perforated plate can be connected to one another, in particular by means of a frame. The further perforated plate can have a number of holes, in particular 20 to 200 holes, each hole having a square shape. The holes of the additional perforated plate can be arranged next to each other and one below the other so that rows of holes are formed.

The holes in the next perforated plate can be formed by means of straight ribs. The webs can have a number of first webs and a number of second webs, wherein the number of first webs are arranged perpendicular to the number of second webs.

The base of the clamping apparatus and/or the base of the fastening apparatus can be arranged between the perforated plate and the further perforated plate.

### BRIEF DESCRIPTION OF THE DRAWINGS

Further advantages and advantageous embodiments of the present disclosure can be found in the figures and the description. All features disclosed in the figures and their descriptions can be substantial to the present disclosure, both individually and in any combination with one another. In the drawings:

FIG. 1 shows an oblique view of a clamping device;

FIG. 2 shows a further oblique view of the clamping device from FIG. 1;

FIG. 3 shows a plan view of the clamping device according to FIG. 1;

FIG. 4 shows an oblique view of a fastening device of the clamping device from FIG. 1;

FIG. 5 shows an oblique view of a clamping apparatus of the clamping device from FIG. 1,

FIG. 6 shows an oblique view of a further exemplary embodiment of a clamping device;

FIG. 7 shows a further oblique view of the clamping from FIG. 6;

FIG. 8 shows an oblique view of a clamping apparatus of the clamping device from FIG. 6;

FIG. 9 shows an oblique view of a further exemplary embodiment of a clamping device;

FIG. 10 shows a further oblique view of the clamping device from FIG. 9;

FIG. 11 shows an oblique view of a fastening apparatus of the clamping device from FIG. 9;

FIG. 12 shows an oblique view of a further exemplary embodiment of a clamping device;

FIG. 13 shows a further oblique view of the clamping device from FIG. 12;

FIG. 14 shows an oblique view of a further exemplary embodiment of a clamping device;

FIG. 15 shows a further oblique view of the clamping device from FIG. 14;

FIG. 16 shows an oblique view of a fastening apparatus of the clamping device from FIG. 14;

FIG. 17 shows an oblique view of a tool holder;

FIG. 18 shows a view of detail XVI according to FIG. 17; and

FIG. 19 shows a further oblique view of the tool holder from FIG. 17.

### DETAILED DESCRIPTION

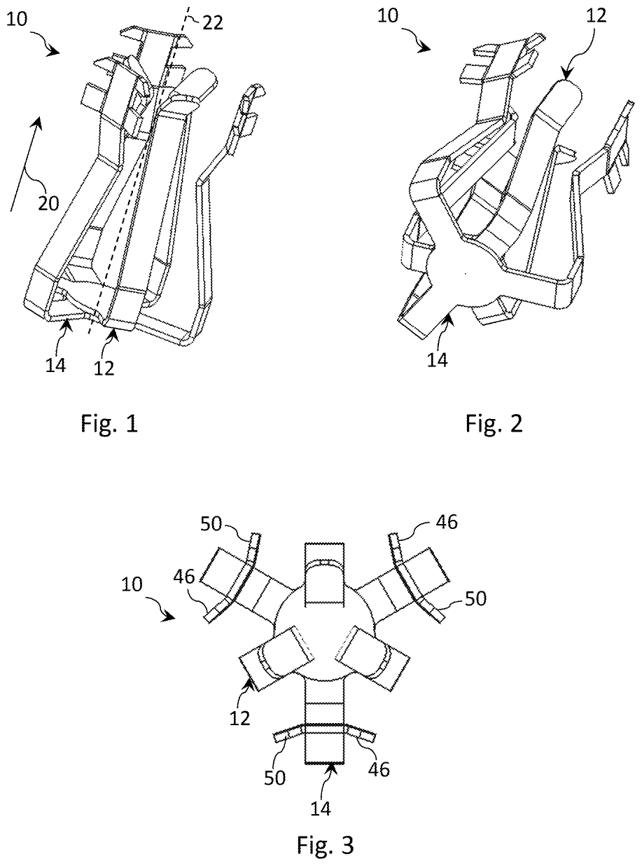

FIGS. 1 to 3 show a clamping device 10 from different viewing directions. The clamping device 10 is designed to hold a surgical tool in a clamped state on a perforated plate.

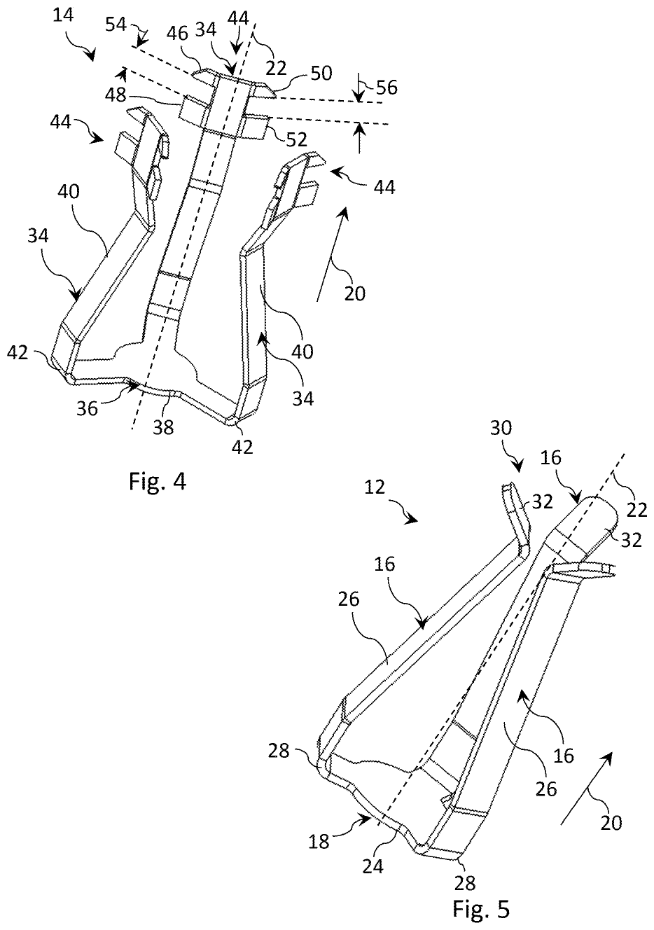

The clamping device 10 has a clamping apparatus 12 and a fastening apparatus 14. For reasons of clarity, the clamping apparatus 12 and the fastening apparatus 14 are shown separately from each other in FIGS. 4 and 5.

The clamping apparatus 12 and the fastening apparatus 14 are two separately designed components. The clamping apparatus 12 and the fastening apparatus 14 are each designed in one piece. The clamping apparatus 12 and the fastening apparatus 14 are each a stamped and bent part.

The clamping apparatus 12 has three clamping springs 16, see FIG. 5. The clamping springs 16 extend away from a base 18 of the clamping apparatus 12 and along a longitudinal direction 20 of the clamping device 10. The longitudinal direction 20 of the clamping device 10 runs parallel to a longitudinal axis 22 of the clamping device 10. The clamping springs 16 are arranged evenly distributed around the longitudinal axis 22.

The base 18 of the clamping apparatus 12 is formed in sections by a circular disk 24. The base 18 of the clamping apparatus 12 is arranged perpendicular to the longitudinal direction 20.

Each clamping spring 16 has a clamping limb 26 for clamping the surgical tool and a spring arc 28. The spring arc 28 is arranged between the base 18 of the clamping apparatus 12 and the clamping limb 26. The clamping limb 26 is designed to contact the surgical tool.

The clamping limbs 26 define an insertion opening 30 for inserting the surgical tool into the clamping device 10.

Each clamping spring 16 has an insertion ramp 32 for inserting the surgical tool into the clamping device 10. The insertion ramps 32 border the insertion opening 30. Each insertion ramp 32 is arranged such that the insertion ramp 32 and the longitudinal direction 20 are at an acute angle to each other. The insertion ramps 32 and the base 18 of the clamping apparatus 12 are arranged at opposite ends of the clamping apparatus.

The surgical tool is inserted or plugged into the clamping device 10 by inserting the surgical tool into the insertion opening 30 and moving the surgical tool in the direction of the base 18 of the clamping apparatus 12. The movement is parallel to the longitudinal axis 22 and opposite to the longitudinal direction 20. The movement brings the surgical tool into contact with the insertion ramps 32. The surgical tool slides along the insertion ramps 32 and thereby pretensions the clamping springs 16 so that the surgical tool is pressed against the other clamping springs 16 by a spring force. The surgical tool can be inserted into the clamping apparatus 12 until it makes contact with the bottom 18 of the clamping apparatus 12. In other words, the base 18 of the clamping apparatus 12 limits the insertion depth of the surgical tool into the clamping device 10.

By inserting the surgical tool into the clamping device 10, the surgical tool is brought into a clamped state. If the clamping device 10 is fastened to the perforated plate, in the clamped state, the surgical tool is fixed to the perforated plate by means of the clamping springs 16 in such a way that the surgical tool is secured against falling out of the clamping device 10 and/or against unintentional displacement relative to the perforated plate.

The fastening apparatus 14 has three fastening springs 34, see FIG. 4. The fastening springs 34 extend away from a base 36 of the fastening apparatus 14 and along the longitudinal direction 20 of the clamping device 10. The fastening springs 34 are arranged evenly distributed around the longitudinal axis 22.

The base 18 of the fastening apparatus 14 is formed in sections by a circular disk 38. The base 38 of the fastening apparatus 14 is arranged perpendicular to the longitudinal direction 20.

Each fastening spring 34 has a fastening limb 40 for fastening the clamping device 10 to the perforated plate and a spring arc 42. The spring arc 42 is arranged between the base 36 of the fastening apparatus 14 and the fastening limb 40. The fastening limb 40 is designed to fasten the clamping device 10 to the perforated plate.

Each fastening limb 40 has a fastening unit 44 for detachably fastening the clamping device 10 to the perforated plate. Each fastening unit 44 has four projections 46, 48, 50, 52. The two projections 46, 48 and the two projections 50, 52 are arranged on opposite sides of the fastening spring 34. Each projection 46, 48, 50, 52 extends in a direction perpendicular to the longitudinal direction 20. Each projection 46, 48, 50, 52 extends radially outwards, see also FIG. 3. Each projection 46, 48, 50, 52 is bent radially outwards.

The two projections 46, 48 are arranged adjacent to each other and are separated by a distance 54 from each other, see FIG. 4. The two projections 50, 52 are arranged adjacent to each other and are separated by a distance of 56. The distance 54 between the two projections 46, 48 and the distance 56 between the two projections 50, 52 is the same. One value of the two distances 54, 56 of the projections 46, 48, 50, 52 is each equal to a value of a thickness of the perforated plate.

This allows the perforated plate to be arranged between the two projections 46, 48 and the two projections 50, 52 while forming a interlocking connection, in particular in the longitudinal direction 20.

To attach the clamping device 10 to the perforated plate, the fastening springs 34 are pretensioned in the direction of the longitudinal axis 22 while reducing the distance between the fastening limbs 40. Subsequently, the fastening apparatus 14 is at least partially inserted into a hole in the perforated plate, and the pretension of the fastening springs 34 is partially released. This causes the fastening limbs 40 to be displaced radially outwards, away from the longitudinal axis 22, and the perforated plate is arranged between the two projections 46, 48 and the two projections 50, 52. The clamping device 10 is fastened, in particular fixed, to the perforated plate by means of the fastening apparatus 14.

In the shown exemplary embodiment from FIGS. 1 to 5, the clamping apparatus 12 and the fastening apparatus 14 are connected to one another, in particular only by means of an integral connection. FIGS. 1 and 2 show that the base 18 of the clamping apparatus 12 and the base 36 of the fastening apparatus 14 are connected to one another by means of the integral connection. In other words, the base 18 of the clamping apparatus 12 and the base 36 of the fastening apparatus 14 are glued together. The integral connection is an adhesive bond.

In the shown exemplary embodiment from FIGS. 1 to 5, the base 18 of the clamping apparatus 12 and the base 36 of the fastening apparatus 14 are connected to one another in such a way that the base 36 of the fastening apparatus 14 forms a first end of the clamping device 10. The insertion opening 30 is arranged at a second end opposite the first end of the clamping device 10.

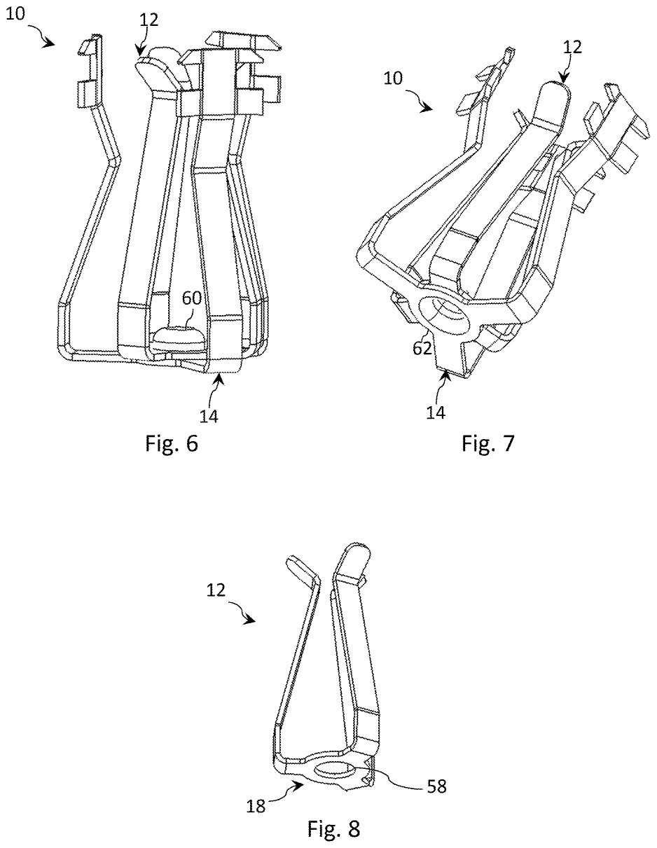

FIGS. 6 to 16 show further exemplary embodiments of the clamping device 10 from FIGS. 1 to 5, wherein the same reference signs are used for identical and functionally equivalent elements and, in this respect, reference can be made to the above explanations regarding the exemplary embodiment from FIGS. 1 to 5 so that substantially only the existing differences will be discussed.

In the illustrated embodiment from FIGS. 6 to 8, the clamping apparatus 12 and the fastening apparatus 14 are connected to one another, in particular only, by means of an interlocking connection. The interlocking connection is formed by means of a deep-drawn connection.

FIG. 8 shows the clamping apparatus 12. The base 18 of the clamping apparatus 12 has a through-hole 58. The through-hole 58 is circular in shape. A portion 60 of the base 36 of the fastening apparatus 14 is driven through the through-hole 58 by deep drawing, in particular without deforming the clamping apparatus 12. The portion 60 driven through the through-hole 58 is deformed in such a way that the portion 60 at least partially rests against the clamping apparatus 12 while forming the interlocking connection.

The deformed portion 60, driven through the through-hole 58, is designed to be mushroom-shaped. On a first side of the base 18 of the clamping apparatus 12, in particular on the side facing away from the insertion opening 30, a portion 62 of the base 36 of the fastening apparatus 14, which is not driven through the through-hole 58, rests against it, see FIG. 7. The portion 60 driven through the through-hole 48 rests against the second side of the base 18 of the clamping apparatus 12, which is opposite the first side and in particular faces the insertion opening 30.

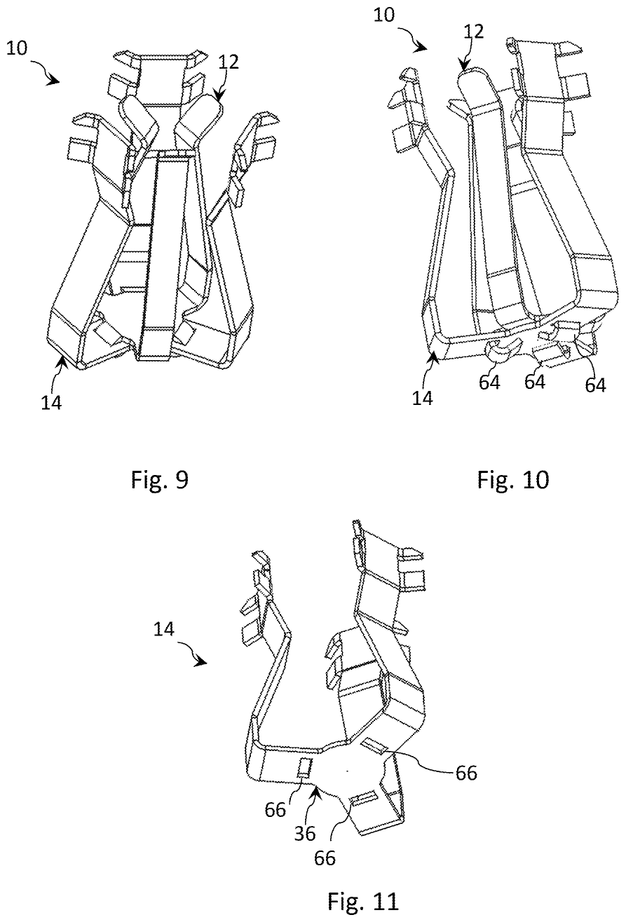

In the shown embodiment from FIGS. 9 to 11, the clamping apparatus 12 and the fastening apparatus 14 are connected to one another, in particular only, by means of a interlocking connection. The interlocking connection is formed by an overlap of the clamping apparatus and the fastening apparatus with each other.

The clamping apparatus 12 has three bending tabs 64, see FIG. 10. The bending tabs 64 are arranged on the base 18 of the clamping apparatus 12. The fastening apparatus 14 has three tab openings 66 for inserting the bending tabs 64. The tab openings 66 are arranged on the base 36 of the fastening apparatus 14, see FIG. 11.

The interlocking connection is created by inserting the bending tabs 64 through the tab openings 66 and then bending the bending tabs 64 over. In other words, the interlocking connection is formed by gripping the fastening apparatus 14, in particular the base 36 of the fastening apparatus 14, with the bent bending tabs 64 of the clamping apparatus 12.

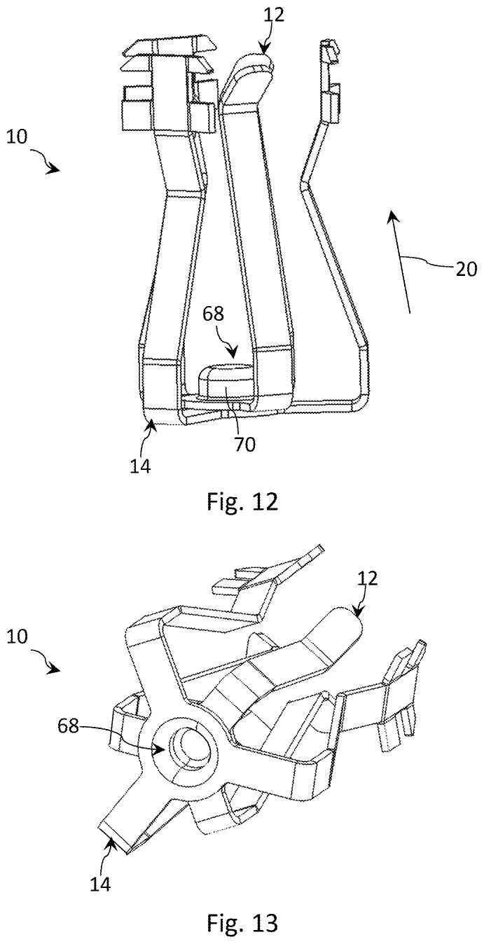

In the shown embodiment from FIGS. 12 and 13, the clamping apparatus 12 and the fastening apparatus 14 are connected to one another, in particular only, by means of a interlocking connection. The interlocking connection is formed by means of a clinch connection 68. The clinch connection 68 is formed by clinching the base 36 of the fastening apparatus 14 and the base 18 of the clamping apparatus 12. The two bases 18, 36 have a dome-shaped portion 70. The clinch connection 68 is formed within the dome-shaped portion 70. The dome-shaped portion 70 extends in the longitudinal direction 20.

Before the clinch connection 68 is made, the base 36 of the fastening apparatus 14 and the base 18 of the clamping apparatus 12 are formed flat. The clinch connection 68 is produced by arranging the flat base 36 of the fastening apparatus 14 on the flat base 18 of the clamping apparatus 12 and subsequently inserting a punch into the material of the two bases 18, 36 to deform the two bases 18, 36.

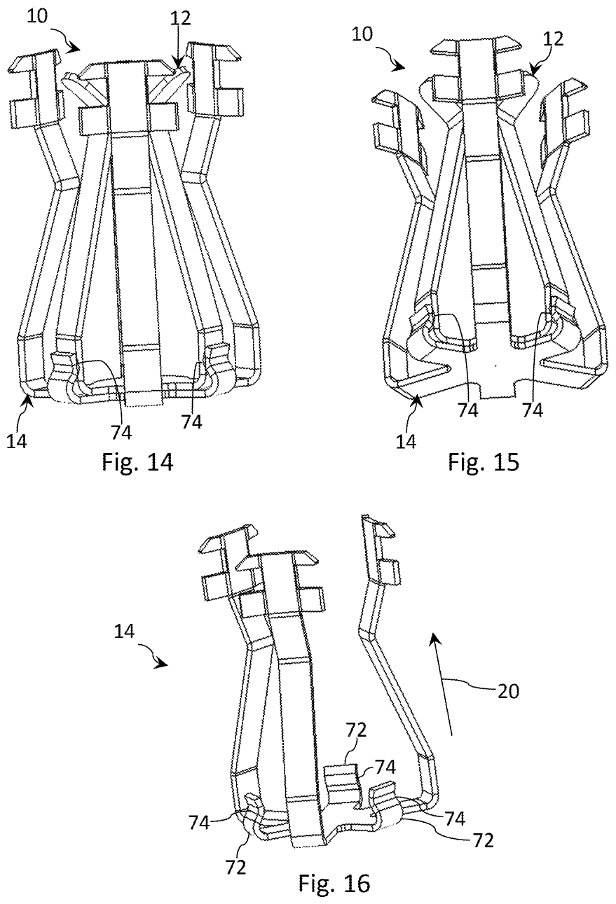

In the illustrated embodiment from FIGS. 14 to 16, the clamping apparatus 12 and the fastening apparatus 14 can be connected to one another, in particular only, by means of a interlocking connection. The interlocking connection can be formed by means of a locking connection.

The fastening apparatus 14 has three first locking elements 72, see FIG. 16. The first locking elements 72 are arranged on the base 36 of the fastening apparatus 14. The first locking elements 72 extend in the longitudinal direction 20. The first locking elements 72 each have a nose-shaped portion 74. Each nose-shaped portion 74 extends in a radially inward direction.

The clamping apparatus 12 can have three second locking elements corresponding to the first locking elements 72. Every second locking element can be a locking opening into which the nose-shaped portion 74 of the particular first locking element 72 can engage while forming the locking connection.

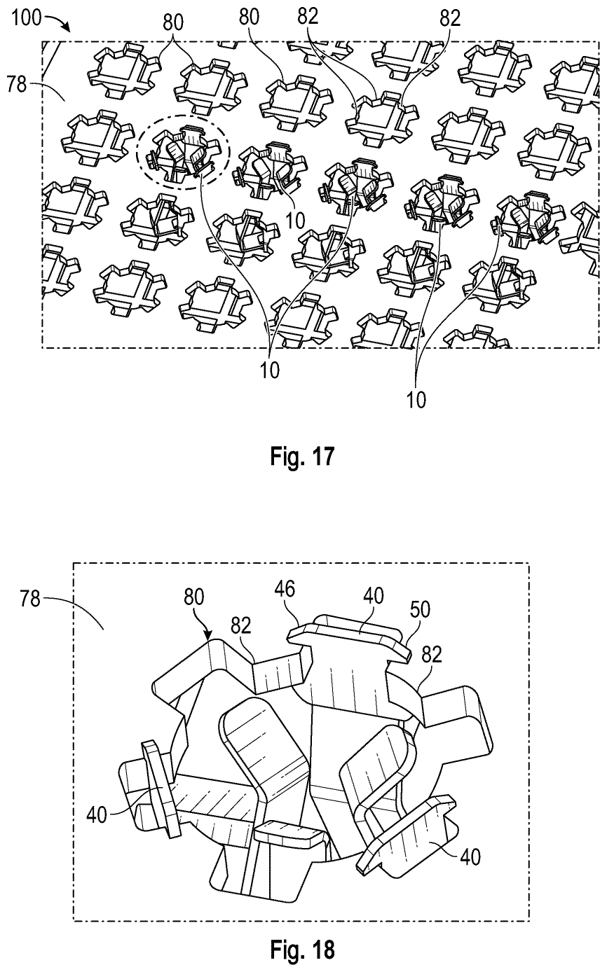

FIG. 17 shows a tool holder 100 for supporting the surgical tool in a designated position. The tool holder 100 has a perforated plate 78 with a multitude of holes 80. Each hole 80 is star-shaped. The star-shaped form is created by the teeth 82 of the perforated plate 78, which define the hole 80. Each hole is bordered by six teeth 82 of the perforated plate 78.

The tool holder 100 has a plurality of previously described clamping devices 10, each of which is attached to a hole 80. In the shown exemplary embodiment from FIG. 17, five clamping devices 10 are attached to five holes 80 by way of example.

FIG. 18 shows in detail a hole 80 of the perforated plate 78 and a clamping device 10 of the tool holder 100 fastened to the perforated plate 78. Each fastening limb 40 of the clamping device 10 is arranged between two adjacent teeth 82. The perforated plate 78 is gripped by the fastening unit 44 of the clamping device 10. This means that the clamping device 10 is attached to the hole 80 of the perforated plate 78 by means of the fastening unit 44. The two projections 46, 50 are arranged on one side of the perforated plate 78 and the two projections 48, 52 are arranged on one side of the perforated plate 78 opposite that side.

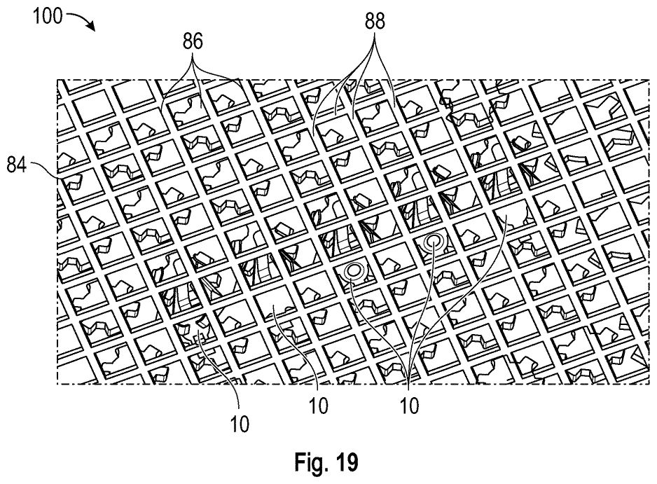

FIG. 19 shows the tool holder 100 from a viewing direction opposite to FIG. 18. The tool holder 100 has a further perforated plate 84 for securing the clamping devices 10 against twisting. The further perforated plate 84 is arranged parallel and offset to the perforated plate 78. The perforated plate 78 and the further perforated plate 84 are connected to one another by means of a frame not shown.

The further perforated plate 84 has a multitude of holes 86. Each hole 86 has a square shape. The holes 86 of the further perforated plate 84 are formed by means of straight webs 88. The holes 86 of the further perforated plate 84 are arranged side by side and among each other in such a way that rows of holes are formed.

The clamping devices 10 are arranged at least partially between the perforated plate 78 and the perforated plate 84. The base 18 of the clamping apparatus 12 and the base 36 of the fastening apparatus 14 are arranged between the perforated plate 78 and the further perforated plate 84.

In FIG. 19, it can be seen that, viewed from left to right by a viewer from FIG. 19, the clamping device 10 from FIGS. 9 to 11, the clamping device 10 from FIGS. 14 to 16, the clamping device 10 from FIGS. 6 to 8, the clamping device 10 from FIGS. 12 and 13 and the clamping device 10 from FIGS. 1 to 5 are fastened to the perforated plate 78.

As the shown and explained exemplary embodiments clearly demonstrate, the present disclosure provides a clamping device and a tool holder that are economical to manufacture.

1. A clamping device for supporting a surgical tool in a clamped state on a perforated plate, the clamping device comprising:
a clamping apparatus; and
a fastening apparatus,
the clamping apparatus comprising a plurality of clamping springs, each clamping spring having a clamping limb for clamping the surgical tool,
the plurality of clamping springs extending from a base of the clamping apparatus in a longitudinal direction of the clamping device,
the clamping limbs defining an insertion opening for inserting the surgical tool into the clamping apparatus,
the fastening apparatus comprising a plurality of fastening springs, each fastening spring having a fastening limb for fastening the clamping device to the perforated plate,
the plurality of fastening springs extending from a base of the fastening apparatus in the longitudinal direction of the clamping device,
each fastening limb comprising a fastening unit for fastening the clamping device to the perforated plate,
the clamping apparatus being formed from a first sheet metal,
the fastening apparatus being formed from a second sheet metal, and
the clamping apparatus and the fastening apparatus being separately designed components that are connected to one another by a frictional, integral and/or interlocking connection.

2. The clamping device according to claim 1, wherein the first sheet metal and the second sheet metal are the same material.

3. The clamping device according to claim 1, wherein the first sheet metal and the second sheet metal have the same thickness.

4. The clamping device according to claim 1, wherein the base of the clamping apparatus and the base of the fastening apparatus are connected to one another by the frictional, integral and/or interlocking connection.

5. The clamping device according to claim 1, wherein at least one of the clamping apparatus and the fastening apparatus comprises a stamped and bent part.

6. The clamping device according to claim 1, wherein:
the clamping apparatus has a first locking element,
the fastening apparatus has a second locking element corresponding to the first locking element, and
the frictional, integral and/or interlocking connection is formed by the first locking element and the second locking element.

7. The clamping device according to claim 1, wherein:
the clamping apparatus has a plurality of bent bending tabs, and
the frictional, integral and/or interlocking connection is formed by gripping the fastening apparatus with the plurality of bent bending tabs.

8. The clamping device according to claim 1, wherein:
the fastening apparatus has a plurality of bent bending tabs, and
the frictional, integral and/or interlocking connection is formed by gripping the clamping apparatus with the plurality of bent bending tabs.

9. The clamping device according to claim 1, wherein the frictional, integral and/or interlocking connection comprises a clinch connection.

10. The clamping device according to claim 1, wherein the frictional, integral and/or interlocking connection comprises a deep-drawn connection.

11. The clamping device according to claim 1, wherein the frictional, integral and/or interlocking connection comprises an upset connection.

12. The clamping device according to claim 1, wherein the frictional, integral and/or interlocking connection comprises an adhesive connection, a welded connection and/or a soldered connection.

13. The clamping device according to claim 1, wherein:
the fastening unit has a first projection and a second projection, and
the fastening unit is configured to accommodate the perforated plate between the first projection and the second projection for fastening the clamping device to the perforated plate.

14. A tool holder for supporting a surgical tool in a designated position, the tool holder comprising:
the clamping device according to claim 1; and
a perforated plate having a hole,
wherein the clamping device is fastened to the hole by the fastening unit.

15. The tool holder according to claim 14, wherein:
the perforated plate has a plurality of teeth that define the hole, and each fastening limb is arranged between two adjacent teeth of the plurality of teeth.

16. The tool holder according to claim 14, further comprising a further perforated plate for securing the clamping device against rotation, the further perforated plate being arranged parallel to and offset from the perforated plate.
